(19) United States
(12) Patent Application Publication
(10) Pub. No.: US 2008/0167616 A1
(43) Pub. Date: Jul. 10, 2008

(54) Device Of An Infusion System

(76) Inventor: Alf Veiby, Nesttun (NO)

Correspondence Address: Carella Byrne Bain Gilfillan;Cecchi Stewart & Olstein, Five Becker Farm Road, Roseland, NJ, 07068, US

(21) Appl. No.: 11/886,121

(22) PCT Filed: Mar. 13, 2006
(86) PCT No.: PCT/NO2006/000093

§ 371 Date: Dec. 17, 2007

(30) Foreign Application Priority Data
Mar. 11, 2005 (NO) 20051278

(51) Int. Cl. A61M 5/148; A61M 5/168

(52) U.S. Cl. 604/134; 604/246

(57) ABSTRACT

The device for an infusion system for delivery of an infusion fluid from a container or bag through a tubing to a user location via a veinflon/cannula has a spring to maintain the pressure in the container/bag so that the fluid flows through the tubing to the user location. The tubing contains a reduction valve for automatic regulation of the fluid pressure to within a desired range and the desired pressure is maintained as long as there is fluid in the container/bag.

The present invention relates to a device for an infusion system for delivery of an infusion fluid from a container, bag or the like, through a tubing to a user location (human being or animal) via a veinflon/cannula, and means to maintain pressure in the bag so that the fluid flows through the tubing to the user location.

The invention also relates to new construction features for a re-sealable box that is used to house the bag with the infusion fluid as described in claim 8, and also the construction of a valve for regulating the pressure in an infusion fluid that is being delivered through a tubing to the object as given in the introduction to claim 10.

The invention also relates to a user system and also an application of the devices for delivery of different forms of fluids that shall be fed into the body of a living creature, especially a human being or an animal.

The reason for such medical treatment can be, for example, that the patient has a general loss of fluid and/or he has a need for a supply of certain nutrients or medicines. Such transfers of fluid to human beings are carried out through the use of an infusion instrument which is called a veinflon. This comprises a cannula that is inserted into the vein and has fastening-wings of a skin-friendly plaster that fastens the veinflon to the patient's skin, and an injection valve which is set in open and closed positions. The other end of the veinflon comprises a space filled with fluid and an opening that makes it possible to connect a tubing that connects the cannula to a bag or container with infusion fluid.

Additionally, the designation infusion fluid can comprise, in this connection, all types of fluids that are to be fed to the body, i.e. to a vein or to other parts of the body.

The fluid transfusions do not require more than that the pressure in the fluid, which is supplied to the vein through a tubing via the cannula, exceeds the pressure in the body/vein. Traditionally, this has been achieved by hanging the bag that contains the fluid, i.e. the bag with infusion fluid, above the patient on a hook on a wall or on a rack that the patient can bring with him when he moves. Normally, the fall-height provides a sufficient infusion pressure. This implies that the patient is tied to the infusion rack, with the associated limited freedom of movement because the patient must bring the rack with him wherever he goes.

Solutions where the patient can easily take the infusion bag with him are also known, and these are particularly favourable in field conditions or catastrophic situations where human beings have a need for different types of emergency assistance. According to known solutions, the device can be carried by an assistant.

Two examples of this type of solution are known from Norwegian Patent No 153,358. This patent describes a pressure infusion pack for pressure infusion of an infusion fluid into the veins of a patient. The pack comprises a filled, flexible bag with infusion fluid with a sealed connection end for connection of an infusion tubing with a regulator and a cannula. These are arranged in a flexible casing together with a flexible, inflatable pressure bag arranged adjacent to the bag of infusion fluid inside the casing. When the pressure bag is inflated it exerts a pressure against the bag of infusion fluid. The fluid will thereby be forced out into the tubing and through the cannula. A regulation of the fluid supply can be brought about in that a drop counter or tubing clamps are incorporated into the tubing.

The Finnish patent FI-109089 describes a similar device for feeding a fluid from an infusion bag. This is comprised of two parts, a box where the bag can be placed and a pressure unit that presses onto the bag.

The disadvantage with these solutions is that they are not self-regulating with regard to the fluid pressure against the veinflon. Therefore, one gets no safeguard against an overpressure being created in the veins into which the content of the infusion bag shall be supplied. Also, the veins can be damaged if the drop counter or tubing clamps are set incorrectly or are fiddled with.

It is an aim of the invention to provide a new construction for delivery of fluids to the body of human beings or animals that, in its entirety, eliminates the above mentioned disadvantages of the previously known solutions.

Consequently, it is an aim of the invention to provide a supply unit that can maintain an approximately constant pressure in the fluid that is delivered to the vein, independently of how much fluid is left in the fluid bag. Thus, it is an aim to provide a storage unit, a box construction whereby the pressure on the bag, and thus the fluids pressure out through the tubing, is held approximately constant.

Furthermore, it is an aim to provide a construction for a reduction valve which ensures that one accurately can control that the fluid pressure to the patient is correct and constant.

It is also an aim of the invention to provide a new infusion unit that is more user-friendly than the known solutions, in that it ought to be easy to bring along, for example, in a pocket, in a waistcoat or in another device close to the body. Thus, this incorporated into a system which ensures that one can easily obtain an individually adapted solution for each patient/user, and which provides the user with a high degree of freedom.

The device (claim 1) according to the present invention is characterised in that the tubing comprises a reduction valve for automatic regulation of the fluid pressure within a desired range, and said desired pressure is maintained as long as there is fluid in the bag. The preferred embodiments appear in the dependent claims 2-7.

The box for storage of the infusion bag is characterised in that the clamping spring is brought to put pressure on the infusion bag via a rigid pressure distribution plate arranged in the box and which covers the whole, or parts, of the surface of the bag, where the plate is preferably as broad as the bag, so that as the bag is squeezed together and is being emptied of fluid, the intermediate plate is pushed downwards and establishes a permanent pressure on the bag. According to a preferred embodiment, the tension spring is an arch-formed tension spring fitted inside the box, as the one end of the tension spring is fixed inside the box while the other end is fastened to a gliding slide which is fitted to an internal surface in the box whereby the tension spring exerts said increasing pressure against the bag at the same time as it is bent so that it becomes flatter whereby the slide is displaced along the inner plate in the box, and the one end of the clamp is preferably fastened to the inside of the lid of the box, while the other end is connected to a slide that can glide along a guide fitted underneath the lid of the box.

The regulating valve according to the invention is characterised in that the valve comprises a first end wall and a second end wall mutually spaced apart in the form of a flexible membrane, wherein a smaller (thinner) tubing of a given length is connected, between and to the membrane and the end wall, respectively, said tubing being arranged to maintain a fluid connection between the tubing area upstream and downstream, respectively, of the valve, via respective openings or channels through the end wall and the membrane, respectively. The preferred embodiments appear in the claims 11-13. By the designation “upstream” one means that the valve is connected into a fluid-carrying tubing so that the flexible membrane turns to the incoming fluid stream, while “downstream” means that the rigid end wall is positioned turned in the other direction, i.e. at the back of the valve.

The system according to the invention is defined in claim 14.

The invention shall now be described in more detail with reference to the enclosed figures, in which:

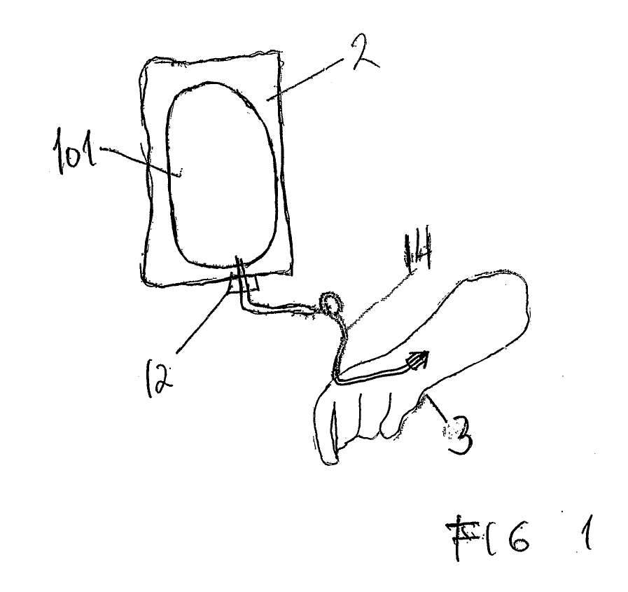

FIG. 1 illustrates how one normally delivers infusion fluid from a bag to a vein in a patient via his lower arm.

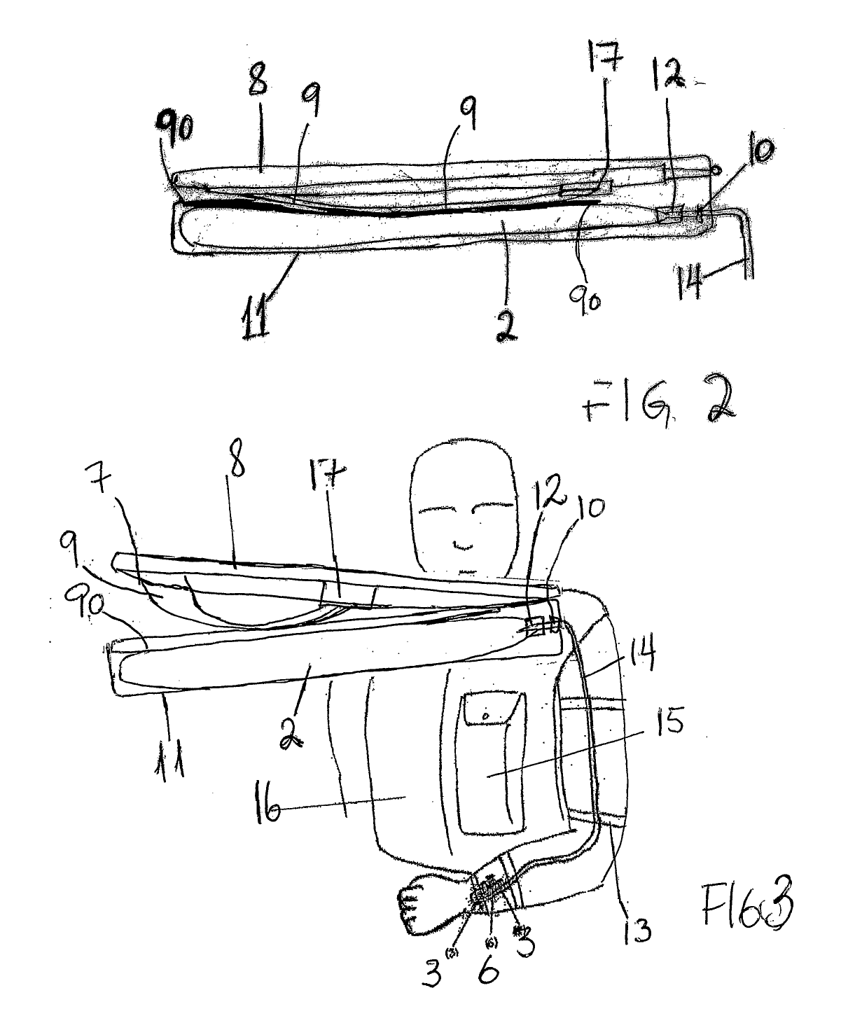

FIG. 2 shows an auto-veinflon box containing an infusion bag and in which a pressure-giving device is arranged.

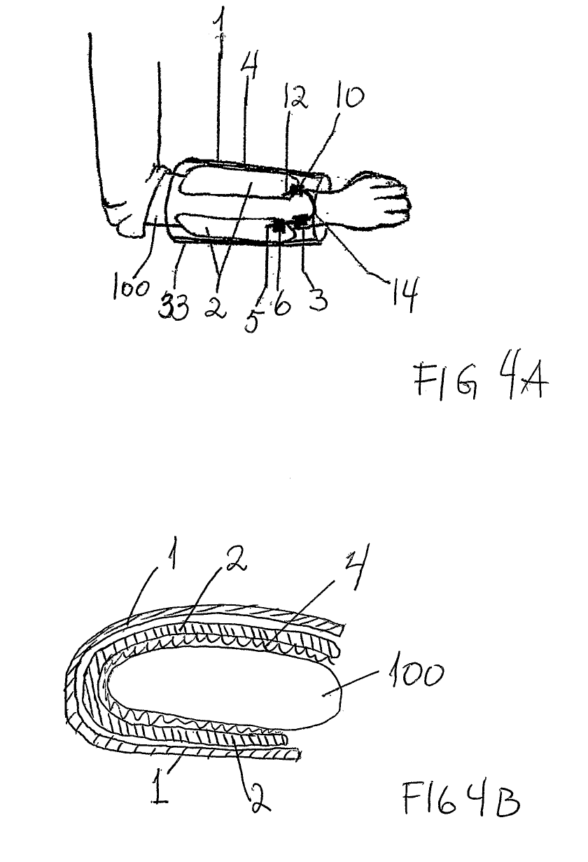

FIG. 3 shows how the auto-veinflon box can be arranged in connection to a waistcoat which is carried by the patient.

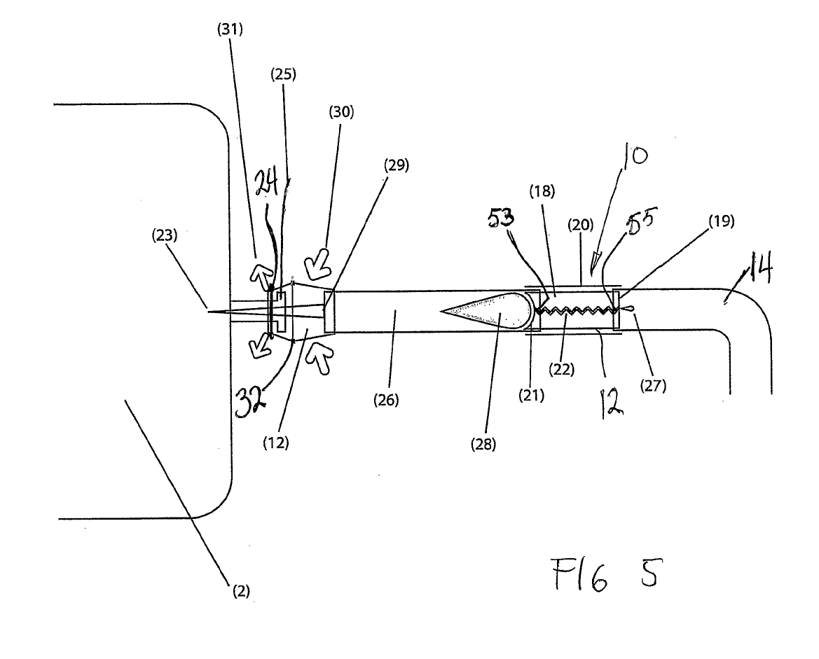

FIG. 4A shows a second embodiment where a lining and a coat are used to provide a fluid pressure inside the bag.

FIG. 4B shows a cross-section of the solution according to FIG. 4A.

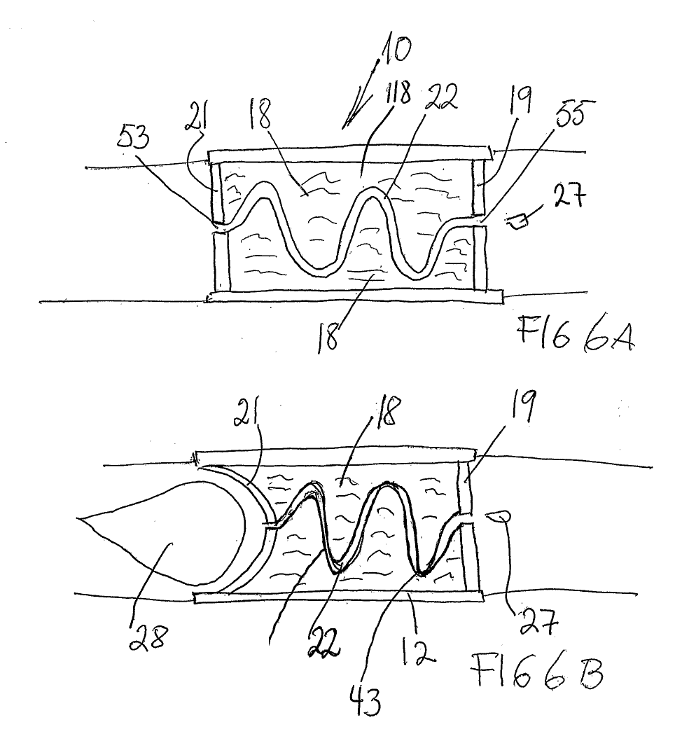

FIG. 5 shows parts of an infusion bag with an automated connection and a reduction valve that is connected to the tubing.

FIG. 6A shows the reduction valve in its inactivated position.

FIG. 6B shows the reduction valve where fluid is flowing in through the valve.

By way of introduction, FIG. 1 shows a bag 2 for infusion fluid, a tubing 14 that brings the fluid from the bag 2 and up to a vein in the lower arm of the patient via a cannula 3 (veinflon). Such a cannula is explained in this text. The bag forms an enclosed space 101 for the fluid. It is this bag that normally hangs on a rack so that sufficient fluid pressure is formed such that the fluid is transferred. An automatic connection 12 is used to connect the tubing 14 to the bag so that the fluid can enter the tubing and further to the cannula/valve 3 in the arm. The automatic connection 12 is well known and comprises a penetration needle which is easily inserted through the bag material in a controlled puncturing of the bag.

According to the invention, the invention comprises a device that can create a fluid pressure for outflow through the tubing. This can be carried out, according to a first example, as it is shown in the FIGS. 2 and 3. Here it can be seen that the infusion bag 2 is arranged inside a box 11. The box comprises a hinged lid 8 which can be opened and closed, and the lid is locked securely in a shut position via a separate locking mechanism. By pressing a button in the lid, the lid is released and can be flipped open again. The box can be transparent so that one can observe the bag and its content from the outside.

The pressure creating body comprises an arch-shaped, broad spring 9 (a blade spring or a hoop) and each end of the clamping spring is fastened underneath the lid. The one end 7 of the pressure clamp 9 is fixed to the underside of the lid 8 while the other end is fixed to a slide 17 that can glide along a guide in the form of a guiding rail fitted to the underside of the lid 8 in the box. The spring is brought to exert a pressure against an infusion bag 2 via a rigid, thin intermediate plate 90 that covers approximately the whole surface of the bag, and which acts as a pressure-distribution plate. The intermediate plate 90 is as wide as the bag area. As the bag 2 is squeezed together and is being emptied of fluid, the intermediate plate is pushed downwards and creates a permanent pressure against the bag. The plate ensures that all the fluid can be driven out of the bag.

At the one end of the box 11 where the tubing 14 shall be inserted to connect to the bag, there is an opening that ensures that one can insert the automatic connection 12. The reduction valve 10 which is connected onto the tubing is arranged adjoining the automatic connection and lies protected inside the box 11. The tubing 14 can be fastened to a person's clothes or arm or other body part, for example, with the help of skin-friendly strips 13. The cannula is normally fastened with skin-friendly plasters 6.

When the bag is placed inside the box, the intermediate plate 90 is laid on top of the bag and the lid is closed and forced in place, the arch-shaped spring 9 will lie on top and press against the plate 90 and the bag 2 so that an increased fluid pressure is created inside the space 101. The pressure clamp 9 will then, at the same time, be bent so that it becomes flatter, and the slide, according to FIG. 2, is displaced towards the right up underneath the lid.

In FIG. 3, the construction is shown enlarged in the forefront of the drawing as a box with an open lid 8 in connection with a user/patient and where the tubing 14 is connected to the veinflon 3 with the cannula 5 on his arm. The box is of such a size that it can be put in the pocket 15 of a shoulder bag 16, a waistcoat or in the breast pocket of a jacket or a shirt. This gives the user increased freedom of movement. When the lid is closed, the pressure distribution plate forces against the bag, and the fluid is forced out of the bag. The pressure against the bag is maintained until the bag is empty of fluid.

An alternative embodiment of the invention is shown in FIGS. 4A and 4B where a fluid filled infusion bag 2 is arranged to enwrap the underarm 100 of a person. The fluid bag 2, which can be placed around the arm in an arch shape (cf. the cross-section in FIG. 4B), is subjected to a pressure so that the fluid is forced out through the tubing 14 and to the veinflon 3 with the cannula 5 in a vein of a person as described above. The fluid can thereby flow into the tubing and to the cannula. Underneath the infusion bag 2, directly on the arm 100, a lining 4 is arranged to protect the skin against direct contact with the plastic material of the infusion bag. Outside and around the bag 2, an elastic protection coat 1 (in particular of a rigid, but flexible plastic) is arranged, and which has such a form that it creates a pressure against the infusion bag. To be placed around the bag, the coat must be partially unfolded under the counteracting influence of the inherent tension force of the coat, be arranged around the underarm, whereupon it creates a necessary pressure against the bag. Thereby, this creates the necessary pressure against the bag 2 and the fluid 101 that is thereby brought to flow out through the tubing.

To maintain the pressure, a band or an elastic band can be arranged outside and round the pressure button according to an alternative solution. As mentioned initially, the cannula is fastened with skin friendly plasters 6. The lining 4 around the elastic band can be fitted to a strip of Velcro 33. The aim of the lining is also to protect against incidental blows against the arm. The infusion bag 2 is connected to the tubing 14 via an automatic connection 12 that is fixed to the tubing. Furthermore, a reduction valve 10 is connected, which ensures that the pressure into the vein does not exceed what is permitted.

An infusion bag 2 is shown in FIG. 5 with automatic connection 12 with a needle 23 that punctures the infusion bag during the fitting where the tubing 14 is connected to the automatic connection 12. The needle 23 itself is fastened to the rear section 29 of the connection. When the automatic connection 30 is clamped on the right side the mouth 31 on the clamp on the left side of the automatic clamp is opened. During the puncturing, a gasket 24 will place itself across a flange 25 at the outlet of the infusion bag. The point 32 makes up a flipping point in the automatic connection. This part of the construction constitutes known technology.

Downstream of the automatic connection 12, a reduction valve 10 is connected to the tubing, which contributes, according to the invention, to adjust the fluid pressure downstream of the valve to a controllable and known level. In addition, an intermediate piece 26 of the tubing 14 between the automatic connection 12 and the reduction valve 10 can be made considerably shorter in that the valve 10 is placed directly in an extension of the automatic connection 12.

The reduction valve 10 can be placed separately as shown in FIG. 5 or placed directly in the automatic connection 12 so that it is tied securely by the latter. It should separately encompass a connection coat—a mantel 20 over the joint. Generally, a reduction valve of a known type can be used.

According to a preferred embodiment, which is described in more detail in the FIGS. 6A and 6B, a reduction valve, which can also be called a throttle valve, can comprise an outer coat 12 of an non-deformable (relatively rigid) body and with an extended casing shape, the end pieces of which are arranged to be connected to the tubing 14. Inside the coat 12, the valve 10 comprises a rigid wall section 19 (the first end wall) at the one end, i.e. at the right side in the figure furthest from the infusion bag 2. The other end of the valve comprises a flexible and elastic membrane 21 that covers the whole of the end surface of the casing, i.e. at the left side of the casing. An opening or channel 53 is formed centrally through the membrane 21, and a corresponding opening/hole is formed centrally through the rigid wall section 19. The membrane can be made from a rubber material, or other suitable, flexible material. In addition, the valve casing and the rigid end wall can be made from a plastic material, the thinner tubing can be a flexible plastic tubing.

A piping or tubing 22 that runs in line with the channel 53 in the elastic membrane is conducted through the rigid chamber 118 in the valve and up to the permanent wall section 19, and secured to run along the inlet of the channel 55 through this section 19. This tubing is thinner than the main tubing 14. The tubing 22 is arranged in large loops through the chamber so that it looks like a sine curve, as shown in the FIGS. 6A and 6B. The chamber 118 is further filled with an elastic material 18 such as a sponge or a porous material or other compressible materials. The looping tubing in the chamber 118 is thus lying surrounded by a bed of the elastic, compressible material 18.

When used, the valve, as can be seen in FIG. 6B, will function such that a higher fluid pressure of the infusion fluid from the bag 2 leads to the flexible membrane 21 being stretched and bent inwards in the chamber 118 in the direction of the rigid wall section 19, so that the membrane 21 forms a cupola-formed or dome-formed shape.

The infusion fluid flows through the opening 53 in the bottom of the membrane 21, in through the tubing 22 and further out on the downstream side of the valve through the channel 55 in the bottom section 19 as the fluid flow shown by the drop 27. When the membrane 21 is forced inwards in the valve in the downstream direction, the tubing is constricted and less fluid is forced through the tubing 22. The flow of fluid is consequently throttled effectively.

A pressure increase in the bag 2 and thereby through the tubing 26, as indicated by the larger drop with the reference number 28 in FIG. 6C, consequently leads to the chamber volume 118 between the membrane and the end section 19 being reduced. The tubing 22 that lies in loops, is squeezed together due to the reduced volume of the chamber 118, and at certain places inside the chamber constrictions on the tubing are made, these are shown by 41 and 43 in FIG. 6B. The elastic material 18 contributes to these constrictions on the tubing 22 being controlled, and without the tubing 22 being so flat in these parts (as can happen, for example, with garden hoses) that the fluid flow ceases completely.

This will also lead to the throttling being effective due to the loops/curves in the tubing limiting/hindering the free passage of fluid through the tubing. That the pressure downstream of the valve is lower than upstream, is indicated by the reference number 27 in FIG. 6B. When the pressure decreases and the membrane pulls back again towards the left and is relieved, the tubing 22 stretches out again towards the more slack loop shape as shown in FIG. 6A.

The restriction against or the throttling of the fluid flow through the membrane 21 and through the tubing 22 decreases continuously with the same rate as the fluid pressure in the bag 2, and further in the tubing section 26, decreases.

When the pressure decreases, i.e. that the bag 2 is being emptied, the membrane 21 is relieved and it returns to its unloaded position with an approximately straight disc-formed shape.

Consequently with this solution one achieves an effective, controlled gradual throttling of the passage of fluid through the tubing. The loops in the tubing and the feature that it is gradually squeezed together will reduce the free passage of fluid. When the fluid pressure from the bag side decreases, the tubing is stretched more and more out to its unloaded, looped position and the fluid flow increases gradually and continuously through the tubing and up to the vein at the correct pressure.

This device is here placed in this way, nearest the infusion bag, either in connection to the automatic connection and inside the box in which the bag is placed to prevent possible fiddling with the valve/apparatus both by the patient himself or if someone should touch it accidentally. Ideally, it should be placed as close to the veinflon/cannula as possible wherever this is placed on the body. In the cases where a drip counter function is required, it should be possible to read this and it should therefore be checked manually. A suitable location for a drip counter is inside an adapted shoulder bag or at a lining on the arm.

It is possible to arrange a signal for alarming when the pressure inside the bag becomes too low. This can be achieved by a slit in the lid where air is let into the box at low pressure. The slit will then emit a piping tone. Another way of ensuring control of the drops is that the fluid passes a constriction, a narrow opening in a membrane, in the tubing where the microscopic expansion is registered. This small movement can be transmitted to a drip counter. The fluid chamber is manufactured traditionally as a plastic bag of a certain quality. The artificial pressure can be elastic band loads in different layers of the bag. This is known technology. It distinguishes itself from the present invention in that this combines controlled pressure in the veinflon at the same time as it is free from other apparatus.

Consequently, with the invention one achieves that an infusion fluid from a container, bag or the like, is delivered through a tubing to a user location (human being or animal) via a veinflon/cannula where the infusion fluid is subjected to a pressure so that it flows through the tubing to the user location, whereby the fluid pressure in the tubing to the user location is regulated in that the fluid is led through a reduction valve which automatically regulates the pressure to a desired value, and said desired pressure is maintained as long as there is fluid in the bag. The connected reduction valve maintains a decreasing resistance against the fluid flow through the tubing in step with the decreasing fluid pressure in the infusion bag as this is being emptied of fluid.

The new throttle valve, according to the invention, can be used for other purposes than for supply of infusion fluid to a patient where there is a need for throttling of a flow of fluid. For example, it can be applied within the food, chemical and pharmaceutical industries. The dimensions of the reduction valve, with regard to length and diameter, and the dimensions of the internal thinner tubing, must be adapted to the actual application area.

1. Device for an infusion system for delivery of an infusion fluid from a bag through a tubing to a user location via a veinflon/cannula, and means to maintain the pressure in the bag so that the fluid flows through the tubing to the user location, characterised in that the tubing comprises a reduction valve for automatic regulation of the fluid pressure to within a desired range, and said desired pressure is maintained as long as there is fluid in the bag.

2. Device according to claim 1 characterised in that the reduction valve comprises a downstream end wall and a flexible membrane arranged upstream, mutually spaced apart, where a smaller tubing of a given length is connected between and to the membrane and the end wall respectively, said tubing maintains a fluid connection between the tubing area upstream and downstream, respectively, of the valve, via respective openings or channels through the end wall and the membrane (21), respectively.

3. Device according to claim 1, characterised in that the space/volume radially outside the tubing, and between the membrane and the end section, respectively, is filled with an elastic material so that the bending of the tubing is controlled and gives the desired throttling of the flow through the tubing.

4. Device according to claim 1 characterised in that the infusion system comprises a clamping arrangement to create said pressure, where the infusion bag is set up to be arranged snugly around a body part/arm and with an over-lying and externally-lying coat that can be tightened and provide a pressure effect against the bag.

5. Device according to claim 1 characterised in that the infusion bag is set up to be arranged in a portable box with a lid, whereby the pressure is maintained by a tension spring arranged inside the box that presses against the bag when the lid is closed.

6. Device according to claim 5, characterised in that the spring is brought to press against the infusion bag via a rigid pressure distribution plate arranged in the box and which covers the whole of the bag, where the plate is preferably as broad as the bag, so that as the bag is squeezed together and is being emptied of fluid, the intermediate plate is pushed downwards and forms a permanent pressure against the bag.

7. Device according to claim 5 characterised in that an arch-formed tension spring is fitted inside the box, as the one end of the tension spring is fixed inside the box while the other end is fastened to a gliding slide which is fitted to an inner surface in the box, whereby the tension spring exerts said increasing pressure against the bag at the same time as it is bent so that it steadily gets flatter whereby the slide is displaced along the inner surface of the box, and the one end of the clamp is preferably fastened to the inside of the lid of the box while the other end is connected to a slide that can glide along a guide fitted underneath the lid of the box.

8. Device for an infusion system to deliver an infusion fluid from a bag through a tubing to a user location via a veinflon/cannula, where the infusion bag is arranged to be placed in a box that can be closed, whereby the pressure is maintained by a tension spring being fitted inside the box, and which creates a pressure against the bag so that the fluid flows through the tubing to the user location, characterised in that the spring is made to put pressure on the infusion bag via a rigid, pressure distribution plate placed inside the box, that covers the surface of the bag, where the plate as broad as the bag, so that as the bag is squeezed together and is being emptied of fluid, the intermediate plate is pushed downwards and creates a permanent pressure on the bag.

9. Device according to claim 8 characterised in that the tension spring is an arch-formed tension spring fitted inside the box, as the one end of the tension spring is fixed inside the box while the other end is fastened to a gliding slide that is fitted to an inner surface in the box, whereby the tension spring exerts said increasing pressure against the bag at the same time as said spring becomes increasingly flatter, whereby the slide is displaced along the inner surface of the box, and preferably, the one end of the clamp is fastened to the inside of the lid of the box, while the other end is connected to a slide that can glide along a guide fitted underneath the lid of the box.

10. Device for a reduction valve for regulation of the pressure in a fluid that flows through a tubing, where the valve is arranged to be connected to the tubing, characterised in that the valve comprises a first end wall and a second end wall mutually spaced apart in the form of a flexible membrane, where a smaller tubing of a given length is connected between and to the membrane and the end wall, respectively, said tubing being arranged to maintain a fluid connection between the tubing area upstream and downstream, respectively, of the valve, via respective openings or channels through the end wall and the membrane, respectively.

11. Device according to claim 10, characterised in that the space/volume radially outside the tubing, and between the membrane and the end section, respectively, is filled with an elastic material so that the bending of the tubing is controlled and gives the intended throttling of the flow of fluid through the tubing.

12. Device according to claim 10 characterised in that the elastic material is of a type of foam plastic.

13. Device according to claim 10 characterised in that the valve is defined by an outer coat of a non-deformable body shaped in an extended casing, the end pieces of which are arranged to be fitted to the tubing.

14. Infusion system for the delivery of an infusion fluid from one of a container and a bag through a tubing to a user location via a veinflon/cannula, means to maintain pressure in the bag so that the fluid flows through the tubing to the user location, and also means to regulate the pressure in the infusion bag, characterised in that the system is arranged to be used as an individual solution where the box with the bag of infusion fluid is carried freely at the user location.

15. (canceled)

16. (canceled)
